(19) United States
(12) Patent Application Publication
(10) Pub. No.: US 2025/0046453 A1
(43) Pub. Date: Feb. 6, 2025

(54) METHOD AND SYSTEM FOR AUTOMATIC DIAGNOSING PREDISPOSITION TO DISEASE

(71) Applicant: Itzik WILF, Atlit (IL)

(72) Inventor: Itzik WILF, Atlit (IL)

(21) Appl. No.: 18/718,230

(22) PCT Filed: Dec. 12, 2022
(86) PCT No.: PCT/IL2022/051307

§ 371 Date: Jun. 10, 2024

Related U.S. Application Data
(60) Provisional application No. 63/288,613, filed on Dec. 12, 2021.

(51) Int. Cl. G16H 50/20; G06T 17/00; G06V 10/44; G06V 10/764; G06V 10/82; G06V 40/16

(52) U.S. Cl. CPC G16H 50/20; G06T 17/00; G06V 10/44; G06V 10/764; G06V 10/82; G06V 40/168; G06V 40/174

(57) ABSTRACT

A computer-implemented method of training a classifier for diagnosing predisposition to a disease comprises steps of: (a) obtaining a training dataset further comprising data relating to previously expertly diagnosed healthy individuals and individuals suffering from the disease; (b) obtaining features from obtained training dataset; (c) training a feature classifier algorithm on the obtained features. The step of obtaining training dataset comprises obtaining sets of multi-spectral and depth head images of each individual at predetermined angles. The step of obtaining features comprises extracting the features from each image of the set such that a multilayer descriptor is generated. The feature classifier algorithm is trained on the multilayer descriptors extracted from the sets of images relating to the expertly diagnosed individuals.

### FIELD OF THE INVENTION

The present invention relates to machine learning image analysis and, more particularly, to face and head-image-based method and system for diagnosing predisposition to a disease such as a stroke-cerebrovascular accident, a chronic obstructive pulmonary disease, obstructive sleep apnea, depression, asthma.

### BACKGROUND OF THE INVENTION

Obstructive sleep apnea is characterized by recurring episodes of breathing pauses during sleep, greater than 10 seconds at a time, caused by a blockage of the upper airway at the level of the pharynx due to anatomic and functional abnormalities of the upper airway.

Cephalometric analysis has also been proposed as a tool for diagnosing sleep-disordered breathing (SDB) [Finkelstein et al., “Frontal and lateral cephalometry in patients with sleep-disordered breathing,” The Laryngoscope 111, 4:623-641 (2001)]. Lateral and frontal cephalometric radiographs were analyzed in a series of normal patients and those with varying degrees of SDB, and the degrees of narrowing or other unfavorable anatomical changes that may differentiate SDB subjects from normal subjects. SDB was found to be associated with statistically significant changes in several cephalometric measurements.

U.S. Pat. No. 9,402,565 discloses a method of analysis in which a target image is registered to define a plurality of keypoints arranged in sets corresponding to polygons or linear segments in the target image. A database of registered and annotated images is accessed and a polygon-wise comparison between the target image and each database image is employed. The comparison is used for projecting annotated locations from the database images into the target image. The aforesaid method is embodied on the basis of craniofacial complex of a subject such as an X-ray image, a Computerized Tomography image or a Magnetic Resonance image. This technical solution requires complex and expensive equipment.

Hence, there is a long-felt and unmet need to provide a handy way of automatically diagnosing predisposition to a disease such stroke-cerebrovascular accident, a chronic obstructive pulmonary disease, obstructive sleep apnea, depression or asthma based on imaging patient's face appearance without use of X-rays or magnetic fields.

### SUMMARY OF THE INVENTION

It is hence one object of the invention to disclose a computer-implemented method of training a convolutional neural-network (CNN) classifier for diagnosing predisposition to a disease; said method comprising steps of: (a) obtaining a training dataset further comprising data relating to previously expertly diagnosed healthy individuals and individuals suffering from said disease; (b) obtaining features from obtained training dataset; (c) training a feature classifier algorithm on said obtained features.

It is a core purpose of the invention to provide the step of obtaining training dataset comprising obtaining sets of multi-spectral and depth head images of each individual at predetermined angles; said step of obtaining features comprises extracting said features from each image of said set such that a multilayer descriptor is generated; said feature classifier algorithm is trained on said multilayer descriptors extracted from said sets of images relating to said expertly diagnosed individuals.

Another object of the invention is to disclose the step of obtaining test data comprises capturing said head images, interrogating said images from a database and a combination thereof.

A further object of the invention is to disclose the capturing said head images performed by an imaging sensor is configured for capturing a luminance image in visible spectral range in a color representation selected from the group consisting of a RGB model, a CMYK model and a Lab color model.

A further object of the invention is to disclose the step of obtaining features from obtained training dataset comprising detecting predetermined feature points on head surfaces and computing local descriptors in proximity of said predetermined feature points.

A further object of the invention is to disclose the feature classifier based on a convolutional network (CNN).

A further object of the invention is to disclose the CNN comprising a support vector classifier algorithm.

A further object of the invention is to disclose each multilayer descriptor comprising head topographic data and a multispectral appearance data registered to each other.

A further object of the invention is to disclose the topographic and appearance data registered to each other according to a set of predetermined landmarks on said head surface of said individual.

A further object of the invention is to disclose the set of head images of said individual comprising at least one image selected from the group consisting of a left-profile head image, a mid-left head image, a frontal head image, a mid-right head image, and a right profile head image, a tilt-down head image and a tilt-up head image.

A further object of the invention is to disclose the step of obtaining said set of head images at predetermined angles comprising generating a 3D-model of a shape of said individual.

A further object of the invention is to disclose the previous expert diagnostics of said multi-spectral and depth head images performed by qualified experts.

A further object of the invention is to disclose the capturing said face images comprising capturing a patient's face according to a predetermined protocol. The predetermined protocol comprises a procedure selected from the group consisting of a face expression, a head position, a head movement and any combination thereof.

A further object of the invention is to disclose the dataset comprising voice records of an individual; said feature classifier algorithm is applied to obtained voice records.

A further object of the invention is to disclose the disease selected from the group consisting of a stroke-cerebrovascular accident, a chronic obstructive pulmonary disease, obstructive sleep apnea, depression, asthma and any combination thereof.

A further object of the invention is to disclose the obstructive sleep apnea selected from the group consisting of snoring, sleep breathing disorders, hypo ventilation syndrome, central sleep apnea and any combination thereof.

A further object of the invention is to disclose predisposition to said disease is graded according to severity of said disease.

A further object of the invention is to disclose a computer-implemented method of assisting in diagnosing predisposition to a disease. The aforesaid method comprises steps: (a) obtaining data relating to an individual to be diagnosed; (b) obtaining features from said data; (c) classifying obtained features by a feature classifier algorithm trained for diagnosing predisposition to a disease; (d) reporting a grade of said predisposition to said decease.

It is a core purpose of the invention to provide the step of obtaining data comprising capturing a set of multi-spectral and depth head images of each individual at predetermined angles; said step of obtaining features comprises extracting said features from each image of said set such that a multilayer descriptor is generated; said step of classifying said obtained features is applied to said multilayer descriptors extracted from said sets of multi-spectral and depth head images.

A computer-implemented system for assisting in diagnosing predisposition to a disease; said system comprising: (a) an imaging sensor configured for capturing face images of a person to be tested; (b) a processor; (c) a memory storing instructions to said processor to execute steps of: (i) obtaining test data of an individual; (iii) obtaining features from obtained test data; (iv) classifying obtained features by a feature classifier algorithm trained for diagnosing predisposition to said disease; (v) reporting a grade of said predisposition to said disease.

It is a core purpose of the invention to provide the instruction of obtaining test data comprising capturing a set of multi-spectral and depth head images of each individual at predetermined angles; said instruction of obtaining features comprises extracting said features from each image of said set such that a multilayer descriptor is generated; said step of classifying said obtained features is applied to said multilayer descriptors extracted from said sets of multi-spectral and depth head images.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIG. 1 is a schematic diagram of a system for assisting in diagnosing predisposition to a disease;

FIG. 2 is a flowchart of a method of generating a multi-layer image;

FIG. 3 is a flowchart of a method of applying landmarks to face images;

FIGS. 4a to 4d are exemplary photographs captured in visible range of 0.4 to 0.7 μm, short-wave infrared range of 1.0 to 3.0 μm, mid-wave infrared range of 3.0 to 5.0 μm, and long-wave infrared range of 8.0 to 14.0 μm, respectively;

FIG. 5 is a flowchart of a method of generating a multi-layer face appearance descriptor;

FIG. 6 is a flowchart of a method of generating an OSA multi-layer classifier;

FIG. 7 is a flowchart of a method of diagnosing predisposition of the patient to a disease; and

FIG. 8 is a flowchart of a method of determining disease progress/recovery.

### DETAILED DESCRIPTION OF THE INVENTION

The following description is provided, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide methods of training a classifier for diagnosing predisposition to a disease and assisting in diagnosing predisposition to a disease and a system for implementing the same.

The present invention is directed to supervised classification in which a classifier is trained with training dataset. The aforesaid training dataset is labeled by medical experts. According to one embodiment of the present invention, a binary labelling is applied to the training data set (healthy/ill individuals). According to an alternative embodiment of the present invention the data relating to individuals suffering from the disease in different severity are labelled in a corresponding manner, using a finite set of severity levels. The multi-class classifier can predict one of said severity levels. According to an alternative embodiment of the present invention the data relating to individuals suffering from the disease in different severity are labelled according to a continuous scale of severity. The classifier score algorithm (regressor) can predict severity score directly.

In the present invention, local features, representing shape and appearance in multiple points spread over the face/head are used. These multiple points are known as “feature points” or “keypoints”. Specifically, the feature points are detected, and then local descriptors around the feature points are computed. A unified process concurrently optimizing feature location of the feature point and its description is also implementable (ASLFeat, see Z. Lou et al., ASLFeat: Learning Local Features of Accurate Shape and Localization 2020 Computer Vision and Pattern Recognition_pp 6589-6598).

Referring to detecting feature points, the following algorithms can be used: extracting “hand-crafted” features (e.g., local image features/descriptors/sound descriptors, such as SIFT, Local Binary Patterns (LBP), PCA-SIFT), and training a “classical” classifier, such as support vector machine (SVM). SVM is an example, there are other schemes such as Random Forest (RF). Examples of using deep local feature/descriptors are disclosed in:

VGG Image descriptor developed by Oxford Visual Geometry Group (VGG), K. Simonyan, A. Vedaldi, and A. Zisserman. Learning local feature descriptors using convex optimisation. IEEE Transactions on Pattern Analysis and Machine Intelligence, 2014.

On the classifier side, SVM or RF can be replaced by training a neural-net classifier using any of the local features above (classic/deep) as inputs (see simple explanation in https://www.baeldung.com/cs/svm-vs-neural-network).

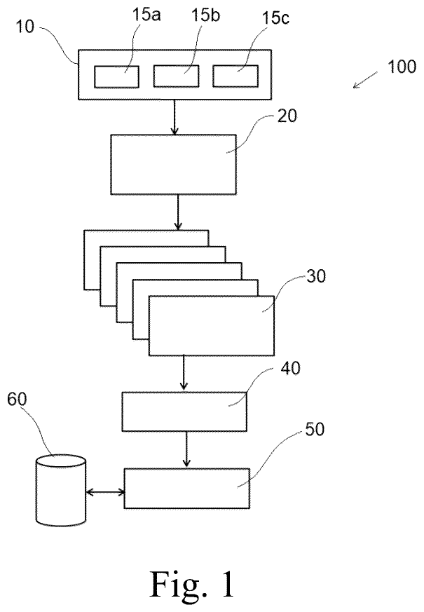

Reference is now made to FIG. 1 presenting a schematic diagram of system 100 for assisting in diagnosing predisposition to a disease. System 100 comprises sensor arrangement 10 including RGB camera 15a, depth camera 15b and thermal camera 15c. Cameras 15a, 15b and 15c are configured for capturing images of patients to be tested.

For example, Intel Realsense 415 camera captures RGBD (RGB+Depth) images at 720p resolution and 30 fps. The RGB map is essentially registered to the depth map based on factory camera calibration. On the basis of detecting landmarks in the RGB image and extracting depth values, a set of RGBZ values is available. Using camera calibration, the landmarks (x, y, Z) where values (x, y) are image coordinates, can be converted in (X, Y, Z) 3D coordinates. By registering a series of (X, Y, Z) of the landmarks to a standardized set of frontal landmarks, frontalization of the appearance and shape data is performed. The frontalization procedure includes uses facial landmarks which can be a set of pre-defined face image locations such as eye corners.

Generally, the landmarks are used for registering images in different spectral ranges because positions of eye corners in the RGB image and thermal image correspond to each other. Specific face locations can be designated as locations where shape and/or appearance data are extractable.

A sound sensor configured for recording patient's voice and breath sounds is also in the scope of the present invention. Sensor arrangement 10 is connected via communication unit 20 to cloud-based medical expert software unit 30. Medical expert software comprises a number of medical expert algorithms configured for analyzing captured images and determining predisposition of the patients to a disease. The results provided by the aforesaid medical expert algorithms are fused in fusion unit 40. The fused results are storable in memory unit 60. Health assessment generator 50 provides reports relating to the tested patients.

The fusion of multiple layers of visual information allows us to combine shape (as inferred from the depth image or derived 3D surface models) with appearance (as captured by the RGB camera), thus learning from data in way that follows human experts. Medical experts in the OSA domain look at the patient and base their indication both on head shape as well appearance (tiredness, depression). The thermal layer provides appearance invisible to the human experts.

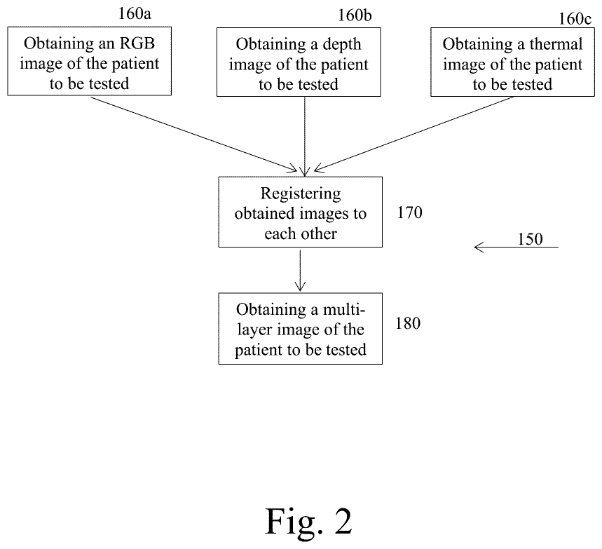

Reference is now made to FIG. 2 presenting a flowchart of method 150 of generating a multi-layer image. An RGB image, a depth image and a thermal image of the patient to be tested are obtained at steps 160a, 160b and 160c, respectively. The aforesaid images can be obtained by photographing the patient or retrieving previously captured images and stored in a memory unit (not shown). The obtained images are registered to each other (step 170) such that an integral multi-layer image is obtained (step 180). It should be emphasized that capturing an integral image combining at least two of the RGB image, depth image and thermal image is also in the scope of the present invention. In the case of using camera Intel Realsense 415, there is no need for registering RGB and depth images. By detecting landmarks in the RGB image and extracting depth value, we obtain a set of RGBD values. Using camera calibration, the landmarks (x, y, Z) values (x, y) are image coordinates, can be converted in (X, Y, Z) 3D coordinates. By registering a series of (X, Y, Z) of the landmarks to a standardized set of frontal landmarks, one can frontalize the appearance and shape data. Effective face frontalization in unconstrained images, Tal Hassner and Shai Harel and Eran Paz and Roce Enbar, 2015 IEEE Conference on Computer Vision and Pattern Recognition (CVPR), {4295-4304}

The integral multi-layer image includes layers R, G, B, T and Z relating to light intensities in red, green, blue and infrared spectral ranges and depth of the captured object, respectively.

This multi-layer image is analyzed in order to determined predisposition to a specific decease such as a stroke-cerebrovascular accident, a chronic obstructive pulmonary decease, obstructive sleep apnea, depression, asthma.

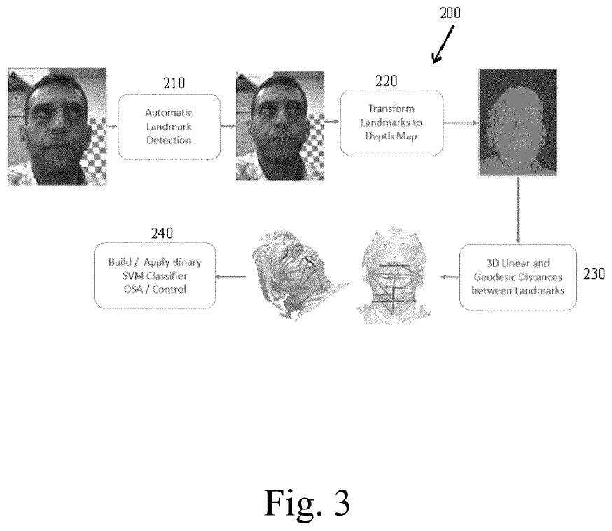

Reference is now made to FIG. 3 presenting a flowchart of method 200 of applying landmarks to face images. As mentioned above, landmarks are automatically detected in captured or previous stored multi-layer images at step 210. Then, landmarks in the multi-layer images are registered to the depth map at step 220.

3D linear and geodesic distances between detected landmarks are calculated (step 230). Finally, a binary SVM classifier of OSA is build or applied based on normalized or relative distances which describe 3D shape, at step 240.

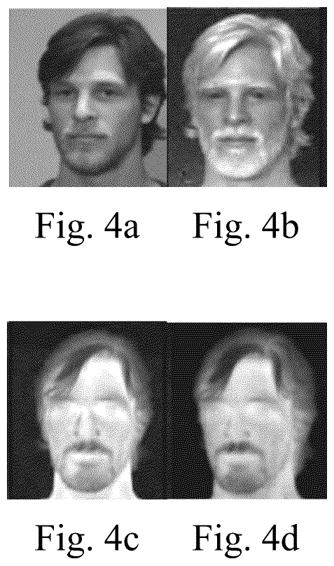

Reference is now made to FIGS. 4a to 4c presenting exemplary photographs captured in different spectral ranges. Specifically, FIG. 4a shows an exemplary photograph captured in visible range of 0.4 to 0.7 μm, FIG. 4b short-wave infrared range of 1.0 to 3.0 μm, FIG. 4c mid-wave infrared range of 3.0 to 5.0 μm, and FIG. 4d long-wave infrared range of 8.0 to 14.0 μm.

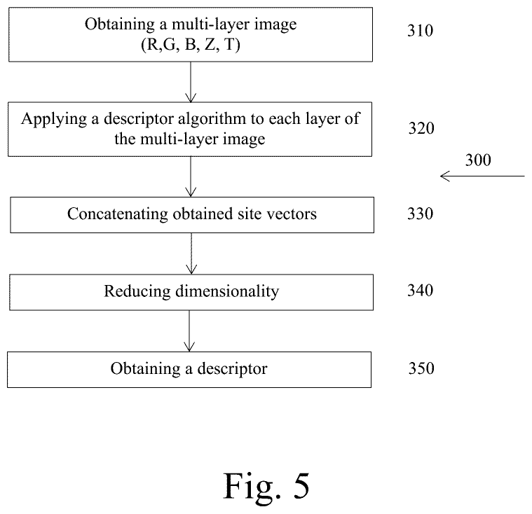

Reference is now made to FIG. 5 presenting a flowchart of obtaining a descriptor. In particular, method 300 of generating a multi-layer face appearance descriptor starts with obtaining a multi-layer image (R, G, B, Z, T) at step 310. Then, local descriptor algorithm is applied to each layer of the abovementioned multi-layer image (step 320). After concatenating obtained site vectors (step 330), the dimensionality is reduced (step 340) such that an integral descriptor is obtained (step 350) which is applicable for determining disposition a patient to a disease.

With the advances in RGBD cameras like Kinect and Realsense, several descriptors combining shape and appearance in a several descriptors are available from prior art, see, for example, Hand-crafted features: E. R. Nascimento, G. L. Oliveira. M. F. M. Campos, A. W. Vieira and W. R. Schwartz, “BRAND: A robust appearance and depth descriptor for RGB-D images,” 2012 IEEE/RSJ International Conference on Intelligent Robots and Systems, 2012, pp. 1720-1726.

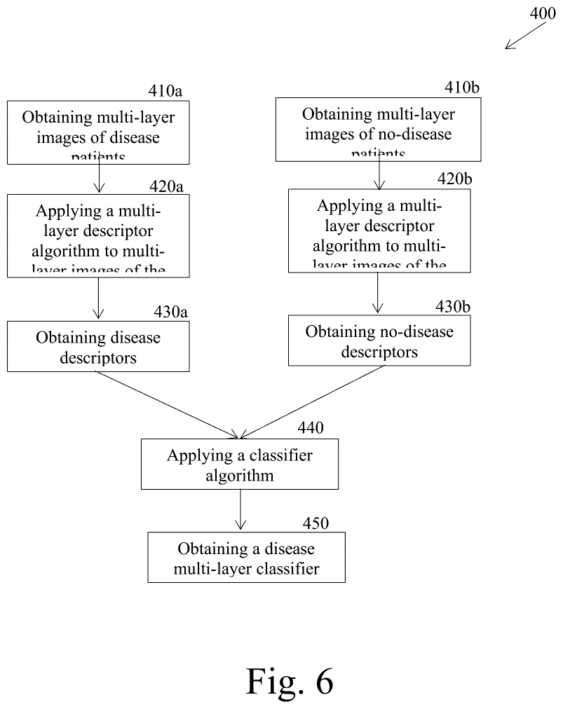

Reference is now made to FIG. 6 presenting a flowchart of method 400 of training a disease multi-layer classifier. Multi-layer images attributed to individuals suffering from a disease such as a stroke-cerebrovascular accident, a chronic obstructive pulmonary decease, obstructive sleep apnea, depression, asthma and healthy individuals are obtained at steps 410a and 410b, respectively. A multi-layer descriptor algorithm is applied to the images belonging to individuals suffering from the aforesaid disease (step 420a) and to the images belonging to the healthy individuals (420b). as a result, a disease descriptor at step 430a and no-disease descriptor 430b are obtained. After applying a classifier algorithm to the obtained descriptors (step 440), a disease descriptor is obtained (step 450).

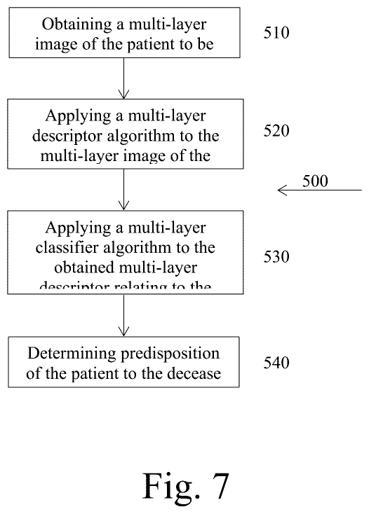

Reference is now made to FIG. 7 presenting a flowchart of method 500 of diagnosing predisposition of the patient to a disease. After obtaining a multi-layer image relating to the individual to be tested (step 510), a multilayer descriptor algorithm is applied to the obtained image (step 520). Then, at step 530, a multi-layer classifier algorithm is applied to the multilayer descriptor obtained at step 520. At step 540, predisposition of the individual to the disease is determined on the basis of obtained multilayer classifier.

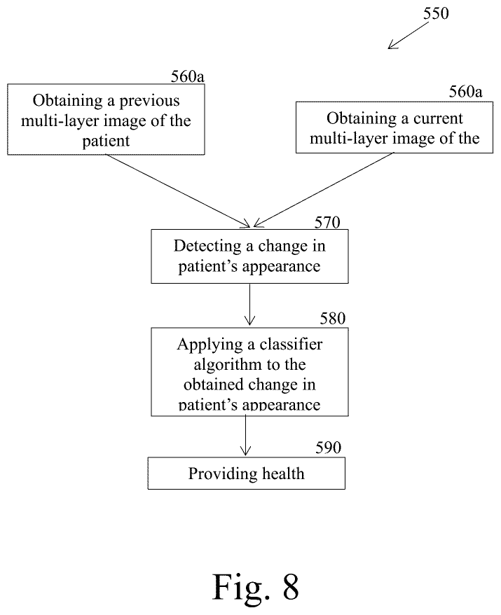

Reference is now made to FIG. 8 presenting a flowchart of method 590 of determining disease progress/remission. At steps 560a and 560b, previous and current multi-layer images of the patient, respectively, are obtained. Numeral 570 refers to the step of detecting differential features between patient's appearances in the aforesaid multi-layer images. Then, a classifier algorithm is applied to the obtained differential features (step 580) such that health indicators characterizing disease progress or remission are reported (step 590).

It should be noted that an arrangement of co-located RGB, depth and thermal cameras captures patient's head from a single viewpoint. A single viewpoint may not capture the full shape and appearance information of the patient. For example, a frontal view, even when augmented by depth information will not fully represent the profile view information.

Several schemes may be used to overcome that limitation:

Capture several distinct views such as left-profile, mid-left, frontal, mid-right, and right frontal, as well as tilt-down and tilt-up. To do so, multiple camera sets can be placed in different positions. For a more practical solution, the patient is asked to turn its head in these directions to be captured by a single arrangement of cameras. To ensure precise head poses and/or guide the patient accordingly, the head pose can be estimated from the RGB or RGBD data as known in prior art.

The extraction of features from the training set, the development of classifiers, and the prediction of disease for the test group, can be iterated for each view. Alternatively, features from 2 or more views can be concatenated into a longer feature vector, optionally undergo a process of dimensionality reduction (such as PCA) and used for training and prediction as described above.

According to another embodiment, the patient is instructed to turn his/her head through the above-mentioned poses in a smooth motion, and the camera arrangement records a sequence of RGBDT images. The sequence is then converted into a 3D surface comprising of a collection of (X, Y, Z, R, G, B, T) tuples.

Several methods of converting such a sequence to a surface are known in prior art—e.g., KinectFusion [R. A. Newcombe et al., “KinectFusion: Real-time dense surface mapping and tracking.” 2011 10th IEEE International Symposium on Mixed and Augmented Reality, 2011, pp. 127-136].

The surface representation is a single complete 3D model of the face & head shape, accompanied with (R, G, B, T) data for each surface points, thus replacing/complementing the (R, G, B, D, T) images captured from multiple viewpoints described above.

To create feature descriptors for classification from such a representation we may follow:

Berretti, Stefano & Werghi, Naoufel & Del Bimbo, Alberto & Pala, Pietro. (2014). Selecting stable keypoints and local descriptors for person identification using 3D face scans. The Visual Computer. 30. 1275-1292. 10.1007/s00371-014-0932-7.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1.-47. (canceled)

48. A computer-implemented method of training a classifier for diagnosing predisposition to a disease; said method comprising steps of:
a. obtaining a training dataset further comprising data relating to previously expertly diagnosed healthy individuals and individuals suffering from said disease;
b. obtaining features from obtained training dataset;
c. training a feature classifier algorithm on said obtained features;
wherein said step of obtaining training dataset comprises obtaining sets of multi-spectral and depth head images of each individual at predetermined angles; said step of obtaining features comprises extracting said features from each image of said set such that a multilayer descriptor is generated; said feature classifier algorithm is trained on said multilayer descriptors extracted from said sets of images relating to said expertly diagnosed individuals.

49. The method according to claim 48, wherein at least one of the following is true:
a. said step of obtaining test data comprises capturing said head images, interrogating said images from a database and a combination thereof;
b. said step of obtaining features from obtained training dataset comprises detecting predetermined feature points on head surfaces and computing local descriptors in proximity of said predetermined feature points;
c. said feature classifier is based on a convolutional network (CNN);
d. each multilayer descriptor comprises head topographic data and a multispectral appearance data registered to each other;
e. said set of head images of said individual comprises at least one image selected from the group consisting of a left-profile head image, a mid-left head image, a frontal head image, a mid-right head image, and a right profile head image, a tilt-down head image and a tilt-up head image;
f. previous expert diagnostics of said multi-spectral and depth head images is performed by qualified experts;
g. said dataset comprises voice records of an individual; said feature classifier algorithm is applied to obtained voice records;
h. said disease is selected from the group consisting of a stroke-cerebrovascular accident, a chronic obstructive pulmonary disease, obstructive sleep apnea, depression, asthma and any combination thereof; and
i. said individuals suffering from said disease expertly graded according to severity of said disease.

50. The method according to claim 49, wherein at least one of the following is true:
a. said capturing said head images is performed by an imaging sensor is configured for capturing a luminance image in visible spectral range in a color representation selected from the group consisting of a RGB model, a CMYK model and a Lab color model;
b. wherein said CNN comprises a support vector classifier algorithm;
c. said CNN comprises a support vector classifier algorithm;
d. said topographic and appearance data are registered to each other according to a set of predetermined landmarks on said head surface of said individual;
e. said step of obtaining said set of head images at predetermined angles comprises generating a 3D-model of a shape of said individual;
f. said capturing said face images comprises capturing a patient's face according to a predetermined protocol; said predetermined protocol comprises a procedure selected from the group consisting of a face expression, a head position, a head movement and any combination thereof;
g. said obstructive sleep apnea is selected from the group consisting of snoring, sleep breathing disorders, hypo ventilation syndrome, central sleep apnea and any combination thereof;

51. A computer-implemented method of assisting in diagnosing predisposition to a disease; said method comprising steps:
a. obtaining data relating to an individual to be diagnosed;
b. obtaining features from said data;
c. classifying obtained features by a feature classifier algorithm trained for diagnosing predisposition to a disease;
d. reporting a grade of said predisposition to said decease;
wherein said step of obtaining data comprises capturing a set of multi-spectral and depth head images of each individual at predetermined angles; said step of obtaining features comprises extracting said features from each image of said set such that a multilayer descriptor is generated; said step of classifying said obtained features is applied to said multilayer descriptors extracted from said sets of multi-spectral and depth head images.

52. The method according to claim 51, wherein at least one of the following is true:
a. said step of obtaining features from obtained data comprises detecting predetermined feature points on head surfaces and computing local descriptors in proximity of said predetermined feature points;
b. said feature classifier is based on a convolutional network (CNN);
c. each multilayer descriptor comprises head topographic data and a multispectral appearance data registered to each other;
d. wherein said set of head images of said individual comprises at least one image selected from the group consisting of a left-profile head image, a mid-left head image, a frontal head image, a mid-right head image, and a right profile head image, a tilt-down head image and a tilt-up head image;
e. said step of obtaining test data comprises capturing said face images, interrogating said images from a database and a combination thereof;
f. said method comprises steps of storing predisposition grade records relating to said individuals in a chronological manner, comparing said predisposition records to each other and generating a disease progress/recovery report;
g. said data comprises voice records of a voice of an individual; said feature classifier algorithm is applied to obtained voice records;
h. said disease is selected from the group consisting of a stroke-cerebrovascular accident, a chronic obstructive pulmonary disease, obstructive sleep apnea, depression, asthma and any combination thereof; and
i. said predisposition to said disease is graded according to severity of said disease.

53. The method according to claim 52, wherein at least one of the following is true:
a. said CNN comprises a support vector classifier algorithm;
b. said topographic and appearance data are registered to each other according to a set of predetermined landmarks on said head surface of said individual;
c. said step of obtaining said set of head images at predetermined angles comprises generating a 3D-model of a shape of said individual;
d. said capturing said face images is performed by an imaging sensor is configured for capturing a luminance image in visible spectral range in a color representation selected from the group consisting of a RGB model, a CMYK model and a Lab color model;
e. said capturing said face images comprises capturing a patient's face according to a predetermined protocol; said predetermined protocol comprises a procedure selected from the group consisting of a face expression, a head position, a head movement and any combination thereof;
f. said disease is selected from the group consisting of a stroke-cerebrovascular accident, a chronic obstructive pulmonary disease, obstructive sleep apnea, depression, asthma and any combination thereof;

54. A computer-implemented system for assisting in diagnosing predisposition to a disease; said system comprising:
a. an imaging sensor configured for capturing face images of a person to be tested;
b. a processor;
c. a memory storing instructions to said processor to execute steps of:
i. obtaining test data of an individual;
ii. obtaining features from obtained test data;
iii. classifying obtained features by a feature classifier algorithm trained for diagnosing predisposition to said disease;
iv. reporting a grade of said predisposition to said disease;
wherein said instruction of obtaining test data comprises capturing a set of multi-spectral and depth head images of each individual at predetermined angles; said instruction of obtaining features comprises extracting said features from each image of said set such that a multilayer descriptor is generated; said step of classifying said obtained features is applied to said multilayer descriptors extracted from said sets of multi-spectral and depth head images.

55. The system according to claim 54, wherein at least one of the following is true:
a. said imaging sensor is configured for capturing a depth image of a face of said person to be diagnosed in a spectral range selected from the group consisting of: 0.4 to 0.7 μm, 1.0 to 3.0 μm, 3.0 to 5.0 μm, 8.0 to 14.0 μm, and any combination thereof;
b. said imaging sensor is configured for capturing a luminance image in visible spectral range in a color representation selected from the group consisting of a RGB model, a CMYK model and a Lab color model;
c. said memory comprises instructions to storing predisposition records relating to said individuals in a chronological manner, comparing said predisposition records to each other and generating a disease progress/recovery report;
d. said instruction of obtaining features from obtained data comprises detecting predetermined feature points on head surfaces and computing local descriptors in proximity of said predetermined feature points;
e. said feature classifier is based on a convolutional network (CNN);
f. each multilayer descriptor comprises head topographic data and a multispectral appearance data registered to each other;
g. said set of head images of said individual comprises at least one image selected from the group consisting of a left-profile head image, a mid-left head image, a frontal head image, a mid-right head image, and a right profile head image, a tilt-down head image and a tilt-up head image;
h. said data comprises voice records of a voice of an individual; said feature classifier algorithm is applied to obtained voice records;
i. said disease is selected from the group consisting of a stroke-cerebrovascular accident, a chronic obstructive pulmonary disease, obstructive sleep apnea, depression, asthma and any combination thereof; and
j. said predisposition to said disease is graded according to severity of said disease.

56. The system according to claim 55, wherein said CNN comprises a support vector classifier algorithm.

57. The system according to claim 55, wherein said topographic and appearance data are registered to each other according to a set of predetermined landmarks on said head surface of said individual.

58. The system according to claim 55, wherein said step of obtaining said set of head images at predetermined angles comprises generating a 3D-model of a shape of said individual.

59. The system according to claim 55, wherein said obstructive sleep apnea is selected from the group consisting of snoring, sleep breathing disorders, hypo ventilation syndrome, central sleep apnea and any combination thereof.
